(19) United States
(12) Patent Application Publication
(10) Pub. No.: US 2023/0321601 A1
(43) Pub. Date: Oct. 12, 2023

(54) Using Water Ponds for Capturing Dioxide and Growing Algae

(71) Applicant: Saudi Arabian Oil Company, Dhahran (SA)

(72) Inventors: Fahd Ibrahim AlGhunaimi, Dhahran (SA); Hassan Alqahtani, Dammam (SA); Mustafa Alkhowaildi, Safwa (SA); Ameerah Bokhari, Thuwal (SA)

(21) Appl. No.: 18/190,660

(22) Filed: Mar. 27, 2023

Related U.S. Application Data
(60) Provisional application No. 63/329,983, filed on Apr. 12, 2022.

(51) Int. Cl. B01D 53/84; B01D 53/62; C12M 1/00; C12N 1/12

(52) U.S. Cl. CPC B01D 53/84; B01D 53/62; C12M 21/02; B01D 2258/0283; C12M 43/04; C12N 1/12; B01D 2257/504; C12M 23/18

(57) ABSTRACT

Methods and systems for sequestering carbon dioxide and growing algae can include: producing fluids from a subsurface formation; separating the fluids into hydrocarbons and produced water; transferring the produced water to a treatment pond; transferring the hydrocarbons to a gas fractionation plant; separating the hydrocarbons resulting in a carbon dioxide side stream; and discharging the carbon dioxide side stream into the treatment pond.

### TECHNICAL FIELD

This specification relates to capturing carbon dioxide and growing algae, particularly capturing carbon dioxide from gas fractionation plants.

### BACKGROUND

Production and emission of CO2 from different sources have caused significant changes in the climate. Microalgae have been shown to efficiently remove carbon dioxide through the rapid production of algal biomass.

### SUMMARY

This specification describes systems and methods that can be used to capture carbon dioxide that would otherwise be released to the atmosphere from gas fractionation plants and other facilities associated with the production of industrially important hydrocarbons. These systems and methods introduce carbon dioxide side streams, for example from gas fractionation plants, into ponds storing produced water (i.e., the naturally occurring water that comes out of the ground along with oil and gas).

In some aspects, methods for sequestering carbon dioxide and growing algae include: producing fluids from a subsurface formation; separating the fluids into hydrocarbons and produced water; transferring the produced water to a treatment pond; transferring the hydrocarbons to a gas fractionation plant; separating the hydrocarbons resulting in a carbon dioxide side stream; and discharging the carbon dioxide side stream into the treatment pond. These methods can include one or more of the following features.

In some embodiments, methods also include compressing the carbon dioxide side stream before discharge to the treatment pond.

In some embodiments, the carbon side stream is at least 99% carbon dioxide.

In some embodiments, the treatment pond is an unstirred treatment pond.

In some embodiments, the treatment pond is a raceway pond.

In some embodiments, methods also include harvesting algae from the treatment pond.

In some embodiments, methods also include removing sulfate from the produced water.

In some aspects, systems for sequestering carbon dioxide and growing algae include: a gas fractionation plant including a carbon dioxide outlet discharging carbon dioxide at least 99% purity; a produced water pond receiving water generated during production of hydrocarbons from a subsurface reservoir; and a carbon dioxide transfer system including conduits extending from the carbon dioxide outlet to a compressor and from the compressor to the produced water pond. Some systems include one or more of the following features.

In some embodiments, systems also include a hydrocarbon-water separator.

In some embodiments, the carbon dioxide transfer system includes a discharge positioned below a nominal water level of the produced water pond.

In some embodiments, the produced water pond is an unstirred treatment pond.

In some embodiments, the produced water pond is a raceway pond.

These systems and methods take advantage of existing oil and gas industry produced water ponds. The modified ponds help increase the growth of algae, absorb carbon dioxide, reuse waste water (e.g., treat produced water before reinjection or use for irrigation including in some cases by carbon dioxide absorption and removal), and generate valuable materials (e.g., algae for biofuel production or for biofertilizers), in a cost effective way.

This approach uses existing water ponds which normally accumulate wastewater to allow it to evaporate to the atmosphere and put them to use to grow and cultivate microorganisms, such as algae. A microbial analysis done on existing ponds found that they contained about 100,000 cells of bacteria per milliliter (ml). A morphological assessment confirmed the existence of algae in these ponds. Directly feeding carbon dioxide into the ponds from an adjacent gas fractionation plant enhance the growth of desirable microbial species while also providing carbon capture.

The details of one or more embodiments of these systems and methods are set forth in the accompanying drawings and the description below. Other features, objects, and advantages of these systems and methods will be apparent from the description and drawings, and from the claims.

### DESCRIPTION OF DRAWINGS

FIG. 1 is an image of a site with produced water ponds and an adjacent gas fractionation plant.

FIG. 2 is a schematic diagram of a system for sequestering carbon dioxide and growing algae.

FIGS. 3A and 3B are schematics illustrating ponds for use in a system for sequestering carbon dioxide and growing algae.

FIG. 4 is a schematic illustrating ponds for use in a system for sequestering carbon dioxide and growing algae.

Like reference symbols in the various drawings indicate like elements.

### DETAILED DESCRIPTION

This specification describes systems and methods that can be used to capture carbon dioxide that would otherwise be released to the atmosphere from gas fractionation plants and other facilities associated with the production of industrially important hydrocarbons. These systems and methods introduce carbon dioxide side streams, for example from gas fractionation plants, into ponds storing produced water (i.e., the naturally occurring water that comes out of the ground along with oil and gas). This bio-mitigation approach provides an effective and sustainable solution for capturing and recycle carbon dioxide using microalgae. The micro-algae growth can use of water that is not suitable for agriculture due to high salinity or oil and minerals contents.

FIG. 1 is an image of a site with five produced water ponds and an adjacent gas fractionation plant. This site includes a system 100 for sequestering carbon dioxide and growing algae includes the ponds 110 and the gas fractionation plant 112. A produced water conduit 114 (i.e., a conduit for transferring produced water) and a carbon dioxide conduit 114 (i.e., a conduit for transferring carbon dioxide) extend from the gas fractionation plant 112 to one of the ponds 110. Although the system 100 is illustrated with one pond 100, one produced water conduit 114, and one carbon dioxide conduit 114, most systems will use multiple ponds and associated conduits. Although the pond 110 is an unstirred treatment pond, some systems use raceway ponds instead of or in addition to unstirred ponds.

An assessment of a possible prototype system was performed at a gas fractionation plant in Saudi Arabia. The proposed location was chosen based on preliminary assessment with regard to carbon dioxide availability and proximity to feed the algae within the ponds, no technical surveying was performed.

Gas-fractionation plants are installations used for the separation of mixtures of light hydrocarbons into individual, or industrially pure, substances. Gas-fractionation plants are typically part of natural gasoline plants, gas refineries, and chemical and petrochemical processing plants. The capacity of gas-fractionation plants may be as high as 750,000 tons of raw material per year, including natural gasolines (which are produced from natural and refinery gases), petroleum stabilization products, and pyrolysis and cracking gases. The raw materials are composed mainly of hydrocarbons containing one to eight carbon atoms per molecule. The separation of the hydrocarbon mixtures is performed by fractional distillation in column distillers. Carbon dioxide and produced water are two of the side streams produced in some gas-fractionation plants. The gas-fractionation plant 112 produces a carbon dioxide side stream which typically has carbon dioxide of at least _% purity.

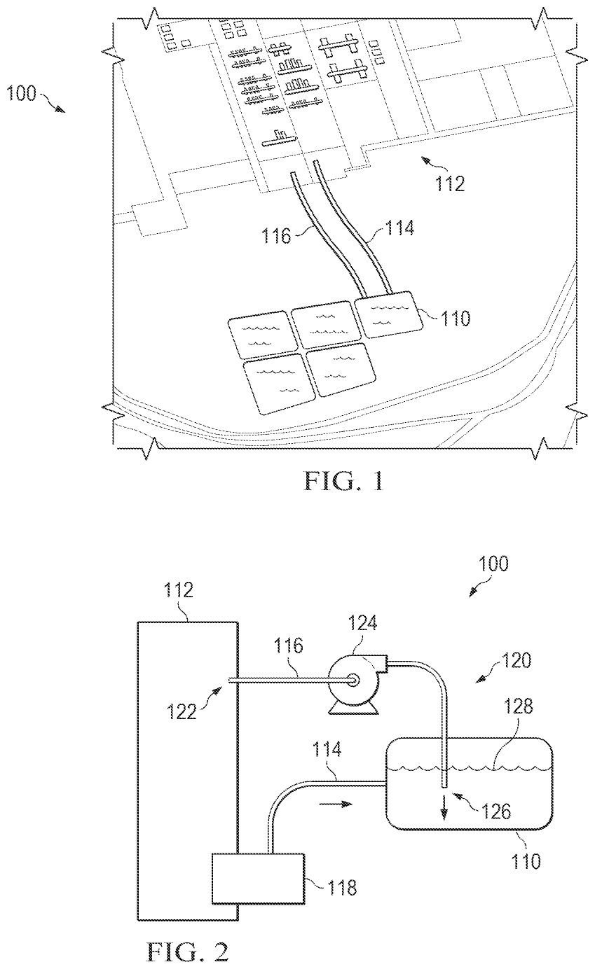

FIG. 2 is a schematic diagram of the system 100 for sequestering carbon dioxide and growing algae in more detail. The plant 112 includes a hydrocarbon-water separator 118 that discharges to the produced water conduit 114 for transfer to the produced water pond 110. The carbon dioxide conduit 116 is part of a carbon dioxide transfer system 120 includes a discharge positioned below a nominal water level of the produced water pond. Other components of the carbon dioxide transfer system 120 include an inlet 122, a compressor 124, and an outlet 126. The conduit 116 includes a portion extending from the inlet 122 to the compressor 124 and a portion extending from the compressor 124 to the outlet 126.

The outlet 126 of the carbon dioxide transfer system 120 is positioned below a nominal water level 128 of the produced water pond 110. Keeping water levels in the pond above the outlet 126 of the carbon dioxide transfer system 120 prevents discharge of carbon dioxide directly into the atmosphere without interaction with the pond water and algae. The system 100 is implemented with a bubbler-based discharge. However. some systems are implemented with other discharges such as diffusers or nozzles. Typically, sufficient produced water is available to maintain the desired water levels. In some implementations, seawater can used in place of or in addition to produced water to maintain water levels in the ponds at nominal levels.

In operation, this approach starts with producing fluids from a subsurface formation. The fluids are separated into hydrocarbons and produced water. This separation takes place at upstream gas plant. The produced water is transferred to a treatment pond and the hydrocarbons are transferred to the gas fractionation plant. Separation of the hydrocarbons results in a carbon dioxide side stream that is discharged the carbon dioxide side stream into the treatment pond. The carbon dioxide can be separated using process technologies including, for example, cryogenic distillation, amine absorption, and membrane separation. The treatment pond can be, for example, an unstirred treatment pond or a raceway pond. Typically, this approach includes compressing the carbon dioxide side stream before discharge to the treatment pond.

As the carbon dioxide bubbles into the pond 110, algae in the pond uses carbon dioxide in the production of biomass (i.e., additional algae). In some implementations, this approach includes harvesting algae from the treatment pond 110 using a bubble generator. The harvested algae can be used for applications such as biofuel production or for biofertilizers. In some ponds, availability of carbon dioxide is the factor limiting algae growth and the other requirements for algae growth (e.g., nutrients) are present in feed water in sufficient quantities that no other materials need to be added to sustain the process. For example, microbial analysis of an existing water pond showed total bacteria presence of 100,000 cell per milliliter.

As part of the continuous efforts to absorb carbon dioxide, new pond designs can be developed. Both unstirred ponds and raceway ponds can be used to implement this approach. These types of ponds have different advantages and challenges but both can provide a suitable environment for algae to grow provided there is a carbon dioxide source feeding the pond.

The ponds 110 shown in FIG. 1 are unstirred ponds. Advantages of using unstirred ponds include low energy consumption as well as low construction and operational costs. The microalgae species are chosen based on local species that can withstand high salinity, high temperatures and a wide range of pH (e.g., Chlamydomonas reinhardtii, Dunaliella salina, Dunaliella tertiolecta, Arthrospira platensis, A. fusiformis, and A. maxima). These species can withstand poor conditions and out compete other microorganisms growing in the same pond. Unstirred ponds are also easy to so scale-up. Some limitations of unstirred ponds include that they can have poor mass and heat transfer sometimes resulting in low productivity. Unstirred ponds also can require monitoring of changing culture conditions, for example, due to seasonal changes, wind, and temperatures changes. Unstirred ponds can be limited to certain types of microalgae species that are capable to grow in poor environmental conditions, and capable to compete with other microorganisms to overcome the common contamination challenges for these systems. Unstirred ponds are not anticipated to be sufficient for human or animal feed applications due to their low yield and susceptibility to contamination.

The approach can also be implemented with raceway ponds. Raceway ponds also have low energy consumption, construction costs, and operational costs. They typically have good mixing for nutrients and heat distribution and are easy to scale-up. Raceway ponds are anticipated to have higher productivity than unstirred ponds. Raceway ponds can require monitoring of changing culture conditions, for example, due to seasonal changes, wind, and temperatures changes.

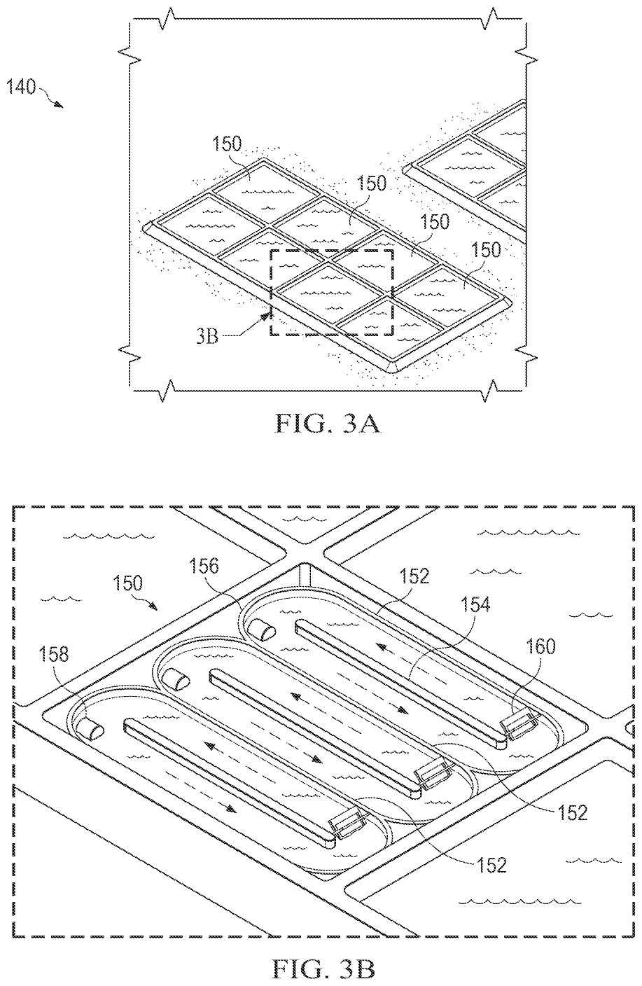

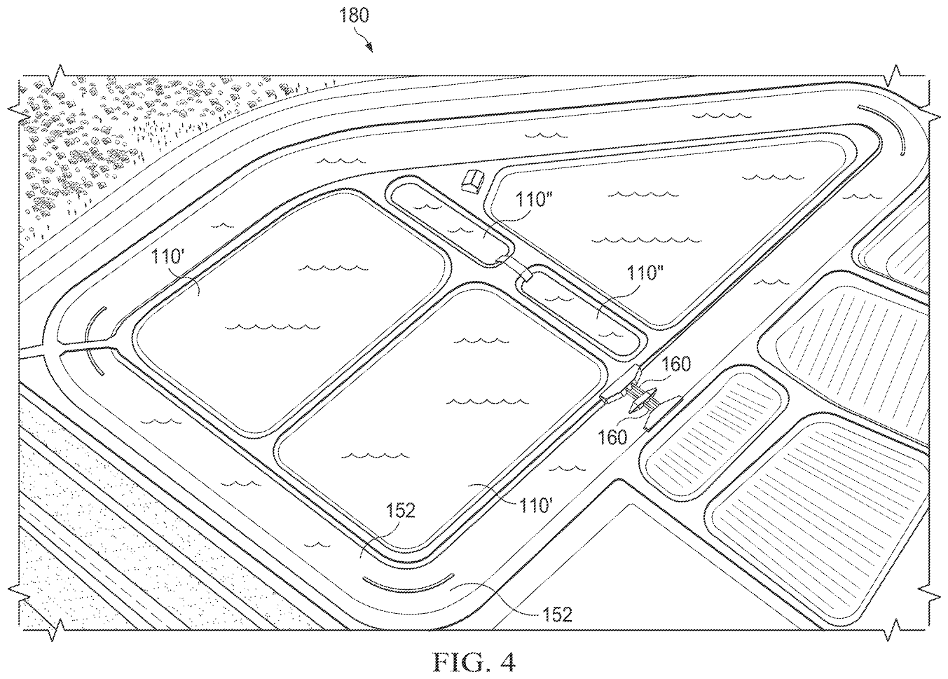

FIGS. 3A and 3B are schematics illustrating a system 140 for sequestering carbon dioxide and growing algae that incorporates raceway ponds 150. The system 140 has eight raceway ponds 150. Some systems have more or fewer raceway ponds and some systems have both raceway and unstirred ponds. Each of the raceway ponds 150 has three raceway channels 152. Each of the raceway channels 152 has a central internal wall 154 and an outer oval wall 156 together defining a raceway shaped oval. An injection pump 158 feeds materials (e.g., water, carbon dioxide, and/or nutrients) into the raceway channel 152. A paddlewheel 160 is operated to circulate water around the raceway channel. Some systems use other approaches to causing circulation such as using nozzles or jets to create a rotary circulation through the channel. The injection pump 158 discharges into the raceway channel 152 in a direction that enhances flow around the channel.

The channels 152 are designed to provide protection from flooding, sedimentation and contamination by pollutants from outside sources. Their dimensions are chosen based upon the available water and planned production level.

FIG. 4 is a schematic illustrating another system 180 for sequestering carbon dioxide and growing algae. The system 180 includes two raceway channels 152, two unstirred ponds 110′ used as facultative ponds, and two unstirred ponds 110″ used as settling ponds. The raceway channels 152 are 3.5 acre raceway channels with associated paddlewheels 160. The infrastructure of the system 80 is based on the system presented by Tryg Lundquist et al. in “Wastewater Reclamation and Biofuel Production Using Algae (DOE & MicroBio project)”.

A number of embodiments of these systems and methods have been described. Nevertheless, it will be understood that various modifications may be made without departing from the spirit and scope of this disclosure. Accordingly, other embodiments are within the scope of the following claims.

What is claimed is:

1. A method for sequestering carbon dioxide and growing algae, the method comprising:
producing fluids from a subsurface formation;
separating the fluids into hydrocarbons and produced water;
transferring the produced water to a treatment pond;
transferring the hydrocarbons to a gas fractionation plant;
separating the hydrocarbons resulting in a carbon dioxide side stream; and
discharging the carbon dioxide side stream into the treatment pond.

2. The method of claim 1, further comprising compressing the carbon dioxide side stream before discharge to the treatment pond.

3. The method of claim 1, wherein the carbon side stream is at least 99% carbon dioxide.

4. The method of claim 1, wherein the treatment pond is an unstirred treatment pond.

5. The method of claim 1, wherein the treatment pond is a raceway pond.

6. The method of claim 1, further comprising harvesting algae from the treatment pond.

7. The method of claim 1, further comprising removing sulfate from the produced water.

8. A system for sequestering carbon dioxide and growing algae, the system comprising:
a gas fractionation plant including a carbon dioxide outlet discharging carbon dioxide at least 99% purity;
a produced water pond receiving water generated during production of hydrocarbons from a subsurface reservoir; and
a carbon dioxide transfer system including conduits extending from the carbon dioxide outlet to a compressor and from the compressor to the produced water pond.

9. The system of claim 8, further comprising a hydrocarbon-water separator.

10. The system of claim 8, wherein the carbon dioxide transfer system includes a discharge positioned below a nominal water level of the produced water pond.

11. The system of claim 8, wherein the produced water pond is an unstirred treatment pond.

12. The system of claim 8, wherein the produced water pond is a raceway pond.
